(19) United States
(12) Patent Application Publication
(10) Pub. No.: US 2019/0392933 A1
(43) Pub. Date: Dec. 26, 2019

(54) System, Method, and Apparatus for Presenting Alternative Therapy

(71) Applicant: AVERLENT CORPORATION, Tampa, FL (US)

(72) Inventor: Bruce Pitcher, Tampa, FL (US)

(73) Assignee: AVERLENT CORPORATION, Tampa, FL (US)

(21) Appl. No.: 16/014,149

(22) Filed: Jun. 21, 2018

(51) Int. Cl. G16H 20/10; G06F 17/30

(52) U.S. Cl. CPC G16H 20/10; G06F 17/30979

(57) ABSTRACT

A system for presenting alternative therapy information to a patient and/or the patient's caregivers (e.g. loved ones, medical staff, etc.) includes a medication manager that keeps track of medications that need to be taken and the time at which the medication is to be taken. Both the patient and caregiver(s) are informed of what needs to be taken and when. After the patient acknowledges taking the medication, the caregivers are also notified. The alternative therapy is presented in such a way as to not disclose any confidential medical information regarding the patient so as to not violate any government rules or laws related to the release of patient information (e.g. complies with HIPPA). Therefore, the alternative therapy is provided in a “blind format,” in that, the advertiser does not know the identity of the patient or caregiver that is receiving/viewing the advertisements.

### FIELD

This invention relates to the field of medicine and more particularly to a system for providing information regarding alternative therapy.

### BACKGROUND

In the field of medicine, there are countless alternative therapies for just about every ailment. There are alternatives for most medications, alternatives for braces, alternatives for therapeutic footwear, alternatives for leg braces, back braces, walkers, etc.

Typically, medical professionals prescribe medications or a medical apparatus based upon familiarity with the medication or apparatus. The medical professional's familiarity initially emanates from advertising provided by the providers of the medications and/or apparatus. Especially pharmaceutical companies—they often have representatives that wine and dine medical professionals to make sure such medical professionals know about the company's products and have some amount of loyalty to that company and or that company's products. Unfortunately, the decisions as to which medication or apparatus are often based almost entirely on these forms of advertisements.

Take for example statins. Statins are a class of lipid-lowering medications and are often prescribed for patients with risks of cardiovascular disease. There are at least seven different statins approved for use in the United States. A given cardiologist will typically prescribe one of the seven statins for any patient with such risk, but that statin may not be the best fit for the patient, as some statins have certain side effects such as muscle pain, muscle damage, liver damage, increased blood sugar, neurological side effects (e.g. numbness), etc. Some statins have more or less side effects based upon the patient as well as other medications that the patient is using.

The patient typically does not question the medical professional and takes the medication as prescribed, but how is the patient to know whether alternative therapies exist? One way is through advertising in the media; television, radio, magazines, billboards, etc. There are many television advertisements for medications and therapeutic devices, but often the patient does not know that they could benefit from an alternate therapy.

Today, if a company, such as a pharmaceutical company that makes one of the statins, knows a particular patient is taking a statin from a different company, the pharmaceutical company could directly advertise to that patient, informing that patient about the benefits of the pharmaceutical company's statin as well as how that companies products counter certain side effects that may be present with that patient. Unfortunately, in the United States, there is a set of laws commonly called HIPPA, which stands for the Health Insurance Portability and Accountability Act. HIPPA provides for stiff penalties for offenses in which medical personnel divulge any private medical information regarding a patient. Therefore, care providers cannot provide information regarding a patient's medications to the pharmaceutical company, as this would infringe upon HIPPA.

What is needed is a system that will provide information regarding alternative therapies to patients, loved ones, and medical practitioners.

### SUMMARY

A system for presenting alternative therapy to a patient and/or the patient's caregivers (e.g. loved ones, medical staff, etc.). A medication manager keeps track of medications that need to be taken and the time at which the medication is to be taken, informing not only the patient of what needs to be taken and when, but also informs others (loved ones, doctors, staff, nurses, etc.) of what needs to be taken, when, and whether the patient acknowledges taking the medication. The alternative therapy is presented in such a way as to not disclose any confidential medical information regarding the patient so as to not violate any government rules or laws related to the release of patient information (e.g. complies with HIPPA). Therefore, the alternative therapy is provided in a “blind format,” in that, the advertiser does not know the identity of the patient or caregiver that is receiving/viewing the advertisements.

In one embodiment, a system for presenting alternate therapies includes a server that has storage for storing medication information and schedules for a patient. The server is connected to a data network. Software running on a caregiver's user device receives as inputs names of medications and schedules for taking the medications. The software running on the user device sends names of the medications and the schedules to the server, where the names of the medications and the schedules are stored in the storage. Software running on the server determines which of the medications is to be taken next and a time that the medication is to be taken. At the time, the server notifies the patient that it is time to take the medication and the server transacts with the caregiver's user device and the caregiver's user device displays a caregiver user interface indicating that it is time for the patient to take the medication to inform the caregiver regarding the medication that is to be taken. Upon taking the medication, the server receives confirmation that the medication was taken and responsive to the confirmation, the server transacts with the caregiver's user device and the caregiver's user device displays a confirmation in the caregiver user interface to inform the caregiver that the medication was taken.

In another embodiment, a method for providing information about an alternate therapy includes accepting inputs containing a name of at least one medication and searching a list of alternate therapies for each of the at least one medication. For each of the at least one medication, if an alternate therapy is found in the list of alternate therapies, presenting information regarding the alternate therapy for that medication.

In another embodiment, method for presenting alternate therapies includes receiving as inputs names of medications and schedules for taking the medications from a caregiver's user device and sending the names of the medications and the schedules to a server through a data network, where the names of the medications and the schedules are stored in a storage. Determining which of the medications is to be taken next and determining a time that the medication is to be taken. At the time, notifying the patient that it is time to take the medication and the server transacting with the caregiver's user device, operating the caregiver's user device to display a caregiver user interface indicating that it is time for the patient to take the medication. If there is an alternate therapy for the medication in an alternate therapy list, the server transacting with the caregiver's user device, operating the caregiver's user device to display information regarding the alternate therapy in the caregiver user interface. Upon taking the medication by the patient, receiving a confirmation from the patient that the medication was taken and the server receiving the confirmation and responsive to receiving the confirmation, the server transacting with the caregiver's user device, operating the caregiver's user device to display the confirmation in the caregiver user interface.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a data connection diagram of the system for presenting alternative therapies.

FIG. 2 illustrates a schematic view of a typical smartphone.

FIG. 3 illustrates a schematic view of a typical computer system such as a server or personal computer.

FIG. 4 illustrates an exemplary user interface for entering a medication schedule.

FIG. 5 illustrates an exemplary user interfaces for informing a patient that it is time to take a medication.

FIGS. 6 and 7 illustrate exemplary user interfaces for informing a caregiver if and when the medication was taken.

FIGS. 8 and 9 illustrate another exemplary method of informing a patient that it is time to take their medication.

FIG. 10 illustrates an exemplary user interfaces for informing a patient that it is time to take a medication with an alternate therapy recommendation.

FIGS. 11 and 12 illustrate exemplary user interfaces for informing a caregiver if and when the medication was taken medication having an alternate therapy recommendation.

FIG. 13 illustrates an exemplary user interface for entering a medication schedule including a drug interaction warning.

FIG. 14 illustrates an exemplary user interface for informing a caregiver if and when the medication was taken including a drug interaction warning.

FIG. 15 illustrates an exemplary data record from the US Food and Drug Administration (FDA) database.

FIG. 16 illustrates an exemplary alternate therapy schedule.

FIG. 17 illustrates an exemplary program flow for provisioning of the system for presenting alternative therapy with patients, medication schedules, and caregiver contacts.

FIG. 18 illustrates an exemplary program flow for informing patients and caregivers when it is time to take medications, the patient using an application to be notified.

FIG. 19 illustrates an exemplary program flow for informing patients and caregivers when it is time to take medications, the patient being called by telephone when it is time to take a medication.

FIG. 20 illustrates an exemplary program flow for informing patients and caregivers when it is time to take medications, the patient using an application to be notified and the caregiver receiving alternate therapy information.

FIG. 21 illustrates an exemplary program flow for informing patients and caregivers when it is time to take medications, the patient being called by telephone when it is time to take a medication and the caregiver receiving alternate therapy information.

FIG. 22 illustrates an exemplary program flow for provisioning of the system for presenting alternative therapy with patients, medication schedules, and caregiver contacts including drug interaction warnings.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

Throughout this description, the term, “patient” describes a person who is receiving a therapy. For example a parent or relative that requires daily medications.

Throughout this description, the term, “caregiver” describes any person who has a vested interest in the wellbeing of the patient. For example, an adult child of the parent that requires daily medications would be a caregiver.

The description uses the term “national drug code directory” is used to describe a database provided by a health organization that contains information regarding medications that are approved for use in a particular nation or territory. For the United States, the national drug code database is provided by the Food and Drug Administration (FDA).

The description uses the term “national drug interaction directory” is used to describe a database provided by a health organization that contains information regarding interactions between medications that are approved for use in a particular nation or territory. For the United States, the national drug code database is provided by the National Institute of Heath (NIH).

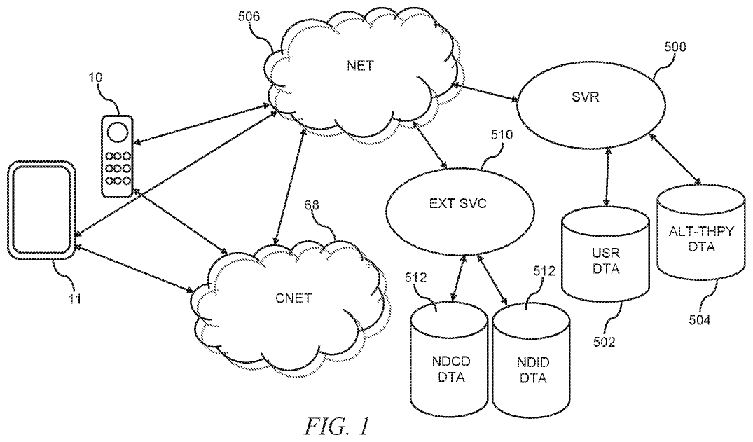

Referring to FIG. 1 illustrates a data connection diagram of the exemplary system for presenting alternative therapy. In this example, one or more devices such as smartphones 10 and tablets 11 communicate through the cellular network 68 and/or through a wide area network 506 (e.g. the Internet) to a server computer 500.

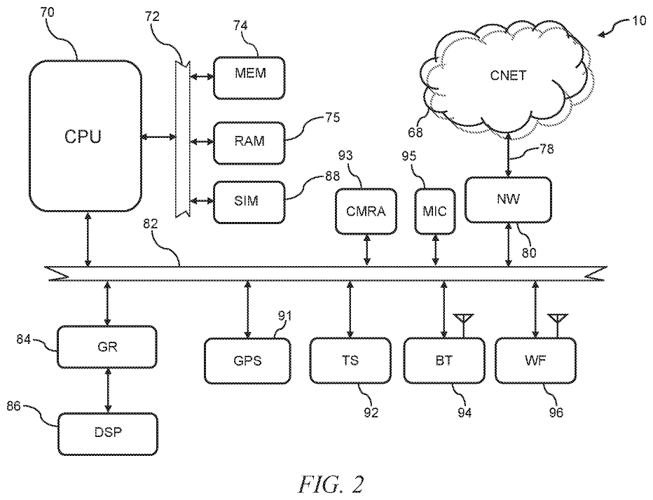

The server computer 500 has access to data storage 502 that is used to store patient data such as medication schedules, primary/secondary contacts, phone numbers, etc. Although one path between the smartphones 10 and tablets 11 and the server 500 is shown going through the cellular network 68 and the wide area network 506 as shown, any known data path is anticipated. For example, the Wi-Fi transceiver 96 (see FIG. 2) of the smartphone 10 or tablet 11 is used to communicate directly with the wide area network 506, which includes the Internet, and, consequently, with the server computer 500.

The server computer 500 transacts with software running on user devices such as smartphones 10 and/or tablets 11 through the network(s) 68/506. Any user device is anticipated, including smart watches, personal computers, etc. The software (e.g., an application) presents menus to/on the smartphones 10/tablets 11, provides data to the smartphones 10/tablets 11, and communicates information to/from the server such as notifying that it is time to take a specific medication.

The server computer 500 transacts with an application running on the smartphones 10/tablet 11 as needed, for example, when notifying that it is time to take a specific medication.

In FIG. 1, an exemplary data connection diagram of the system for presenting alternative therapy is shown. The system for presenting alternative therapy, for example, includes a server 500 that transacts with one or more end user devices (e.g. smartphones 10 or tablets 11) to manage current therapies and to present alternative therapy.

The system for presenting alternative therapy stores user data (e.g. in a disk drive that is local to the server 500, cloud-based storage, etc.). In such storage is user data 502 such as account information, patient information (e.g. medications, dosages, time to take medications) and alternate therapy data 504 (e.g. information regarding alternate therapy that is to be presented to patient and caregivers, rules data for determining cross-references and when to present alternate therapies) The system for presenting alternative therapy accesses the national drug code directory 512 and national drug interaction directory 513 through an external server 510 (e.g. a server operated by a national organization such as the Federal Drug Administration or National Institute of Health within the United States of America), for example, through the network 506 or delivered/accessed through any known delivery/access method. Any network topology and composition is fully anticipated including only a data network 508 (e.g. the Internet) or only the cellular network 68.

The national drug code directory 512 includes, for example, drug categories and families that are used to select alternative therapies, as well as drug descriptions (e.g. type of pill/package) that is used to inform the patient which pill to take, and, optionally. The national drug interaction directory 513 includes drug interaction information that, optionally, is used to warn patients and caregivers of potential drug interaction issues.

Referring to FIG. 2, a schematic view of a typical end-user device, a smartphone 10 is shown though other end-user devices such as a tablet 11, smart televisions, connected televisions, personal computer, etc., are fully anticipated. Although any end-user device is anticipated, for clarity purposes, a smartphone 10 will be used in the remainder of the description.

The system for system for presenting alternative therapy is described using a processor-based end-user device (e.g., smartphone 10) for providing the login and user interfaces necessary for managing patient information and for presenting alternative therapy. The present invention is in no way limited to using a smartphone 10 and any similar device is anticipated (e.g., cellular phone, portable digital assistant, tablet, notebook, etc.).

The example smartphone 10 represents a typical device used for accessing user interfaces of the system for presenting alternative therapy. This exemplary smartphone 10 is shown in one form with a sample set of features. Different architectures are known that accomplish similar results in a similar fashion and the present invention is not limited in any way to any particular smartphone 10 system architecture or implementation. In this exemplary smartphone 10, a processor 70 executes or runs programs in a random-access memory 75. The programs are generally stored within a persistent memory 74 and loaded into the random-access memory 75 when needed. Also accessible by the processor 70 is a SIM (subscriber information module) card 88 having a subscriber identification and often persistent storage. The processor 70 is any processor, typically a processor designed for phones. The persistent memory 74, random-access memory 75, and SIM card are connected to the processor by, for example, a memory bus 72. The random-access memory 75 is any memory suitable for connection and operation with the selected processor 70, such as SRAM, DRAM, SDRAM, RDRAM, DDR, DDR-2, etc. The persistent memory 74 is any type, configuration, capacity of memory suitable for persistently storing data, for example, flash memory, read only memory, battery-backed memory, etc. In some exemplary smartphones 10, the persistent memory 74 is removable, in the form of a memory card of appropriate format such as SD (secure digital) cards, micro SD cards, compact flash, etc.

Also connected to the processor 70 is a system bus 82 for connecting to peripheral subsystems such as a cellular network interface 80, a graphics adapter 84 and a touch screen interface 92. The graphics adapter 84 receives commands from the processor 70 and controls what is depicted on the display 86. The touch screen interface 92 provides navigation and selection features.

In general, some portion of the persistent memory 74 and/or the SIM card 88 is used to store programs, executable code, and data, etc. In some embodiments, other data is stored in the persistent memory 74 such as audio files, video files, text messages, etc.

The peripherals are examples and other devices are known in the industry such as Global Positioning Subsystem 91, speakers, microphones, USB interfaces, camera 93, microphone 95, Bluetooth transceiver 94, Wi-Fi transceiver 96, image sensors, temperature sensors, etc., the details of which are not shown for brevity and clarity reasons.

The cellular network interface 80 connects the smartphone 10 to the cellular network 68 through any cellular band and cellular protocol such as GSM, TDMA, LTE, etc., through a wireless medium 78. There is no limitation on the type of cellular connection used. The cellular network interface 80 provides voice call, data, and messaging services to the smartphone 10 through the cellular network 68.

For local communications, many smartphones 10 include a Bluetooth transceiver 94, a Wi-Fi transceiver 96, or both. Such features of smartphones 10 provide data communications between the smartphones 10 and data access points and/or other computers such as a personal computer (not shown).

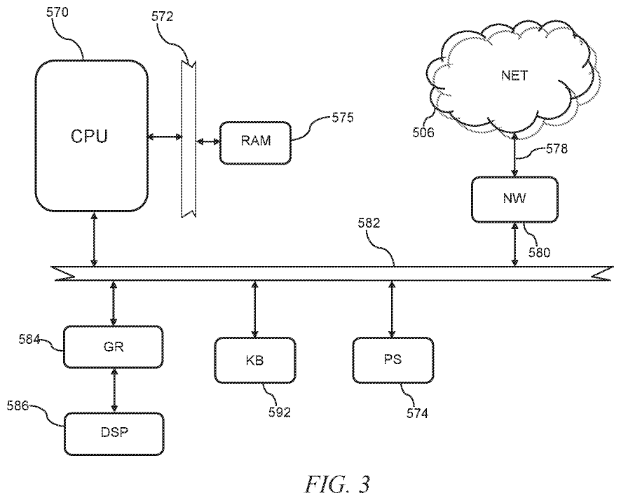

Referring to FIG. 3, a schematic view of a typical computer system (e.g., server 500) is shown. The example computer system 500 represents a typical computer system used for managing patient data and for presenting alternative therapy. This exemplary computer system is shown in its simplest form. Different architectures are known that accomplish similar results in a similar fashion and the present invention is not limited in any way to any particular computer system architecture or implementation. In this exemplary computer system, a processor 570 executes or runs programs in a random-access memory 575. The programs are generally stored within a persistent memory 574 and loaded into the random-access memory 575 when needed.

The processor 570 is any processor, typically a processor designed for computer systems with any number of core processing elements, etc. The random-access memory 575 is connected to the processor by, for example, a memory bus 572. The random-access memory 575 is any memory suitable for connection and operation with the selected processor 570, such as SRAM, DRAM, SDRAM, RDRAM, DDR, DDR-2, etc. The persistent memory 574 is any type, configuration, capacity of memory suitable for persistently storing data, for example, magnetic storage, flash memory, read only memory, battery-backed memory, magnetic memory, etc. The persistent memory 574 (e.g., disk storage) is typically interfaced to the processor 570 through a system bus 582, or any other interface as known in the industry.

Also shown connected to the processor 570 through the system bus 582 is a network interface 580 (e.g., for connecting to a data network 506), a graphics adapter 584 and a keyboard interface 592 (e.g., Universal Serial Bus—USB). The graphics adapter 584 receives commands from the processor 570 and controls what is depicted on a display 586. The keyboard interface 592 provides navigation, data entry, and selection features.

In general, some portion of the persistent memory 574 is used to store programs, executable code, data, and other data, etc.

The peripherals are examples and other devices are known in the industry such as pointing devices, touch-screen interfaces, speakers, microphones, USB interfaces, Bluetooth transceivers, Wi-Fi transceivers, image sensors, temperature sensors, etc., the details of which are not shown for brevity and clarity reasons.

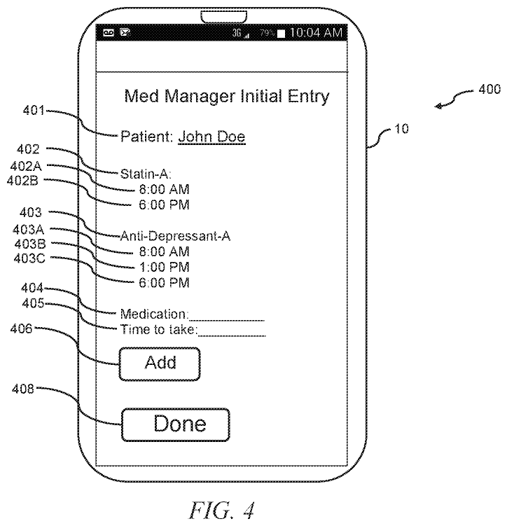

Referring to FIG. 4, a representative user interface for entering medication data 400 of the system for presenting alternative therapy is shown. Again, a smartphone 10 with a smartphone user interface is shown throughout, as is anticipated that user interfaces for other devices such as tablets 11 will differ. It is well known in the industry that applications with user interfaces that contain private or sensitive information typically require user to present credentials before gaining access to the applications. This step is well known and not included here within for brevity and clarity reasons.

As with many such interfaces, the user interface for entering medication data 400 includes a place to enter the name of the user 401 (e.g. John Doe). The user interface for entering medication data 400 is shown partially populated, in that two medications have already been entered; Statin-A 402 and Anti-Depressant-A 403. The time of day to take each medication has also been entered; 8:00 AM 402A and 6:00 PM 402B for the Statin-A 402 and 8:00 AM 403A, 1:00 PM 403B, and 6:00 PM 403C for the Anti-Depressant-A 403. Note that throughout this document, non-trademark names are used to limit confusion but in actual use, brand name or generic names are used to describe the medications (e.g. Lipitor® or aspirin).

The user interface for entering medication data 400 has facilities to add/remove medications. For example, to add a new medication, a user enters the name of the medication 404, the time to take the medication 405 and then select the add feature 406. To remove a medication, the user right clicks on the medication and selects “delete.” To remove only one of the time entries (e.g. 8:00 AM 402A), the user right clicks on the time entry and selects delete. When finished entering medication information for the patient 401, the done feature 408 is invoked to save data and proceed with other user interface screens.

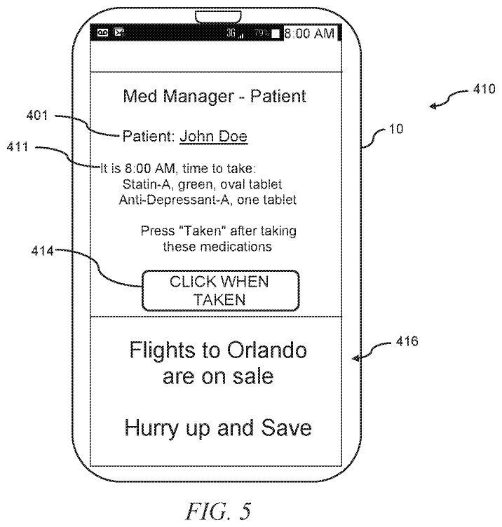

Referring to FIG. 5, an exemplary user interface for informing a patient 410 that it is time to take a medication is shown. In general, once the schedule is established for the patient 401, at each time in the schedule, at the very least, the patient 401 is informed as to what medication is to be taken at the present time. In the example patient user interface 410, it is 8:00 AM and the patient 401 is presented with a message 411 describing the medication that needs to be taken at this time. As shown, in some embodiments, the message 411 includes description information (e.g. Statin-A is a green, oval tablet) and quantity information (e.g. one tablet) are included. Although not shown, it is fully anticipated that multiple individual alerts be provided, one for each medication that needs to be taken, or a single alert listing all medications that need to be taken.

The patient 401 sees a “Click when Taken” directive 414 after the described medications 411. The intent is for the patient 401 to select (e.g. press, tap, click on) the “Click when Taken” directive 414 after taking the medications in the message 411.

It is fully anticipated that, along with the display information presented in this example patient user interface 410, vibration, indicators, noises/tunes are emitted from the smartphone 10 to gain the attention of the patient 401.

In some embodiments, an advertisement 416 is presented, possibly changing each time the patient user interface 410 is displayed. In such, the advertisement is selected from one or more predetermined advertisements and displayed according to an algorithm such as randomly or with some regard to revenue arranged per each advertisement.

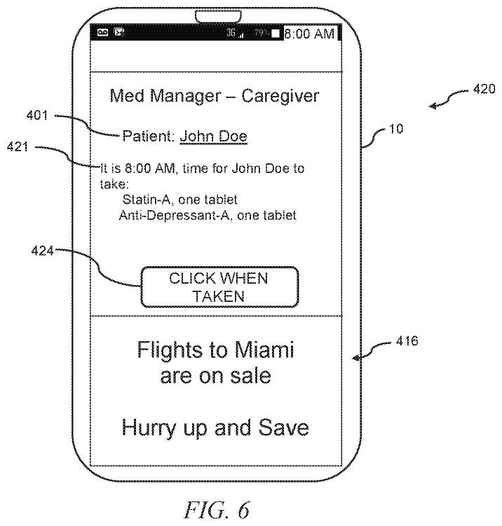

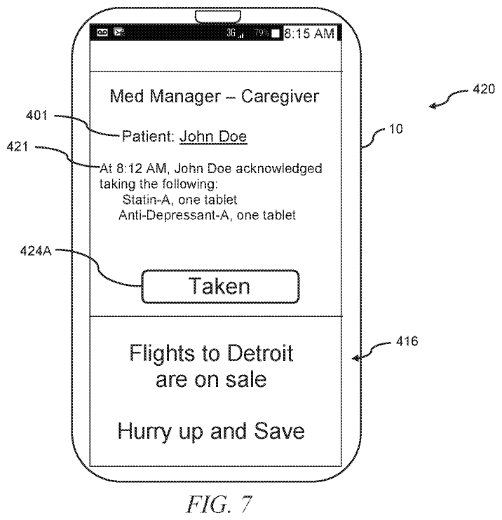

Referring to FIGS. 6 and 7, exemplary user interfaces for informing a caregiver 420 if and when the medication was taken are shown. These user interfaces for informing a caregiver 420 if and when the medication was taken are one way anticipated to provide such information to the caregivers, as it is equally anticipated that based upon caregiver configuration settings, the caregiver will receive text messages, emails, phone calls, etc., at selectable events. Examples of such events are: when the patient 401 is scheduled to take the medication, when the patient 401 takes the medication, when the patient 401 fails to indicate that they have taken the medication for some time after the scheduled time, etc. For example, in one configuration, the caregiver only receives a text message if the patient 401 fails to indicate that they have taken the medication thirty minutes after the scheduled time for that medication.

Many patients 401 have medical or aging issues that make it easy to miss taking their medications when required. The caregiver user interface 420 informs the caregiver (e.g. loved one, medical personnel) that the medication is due to be taken and has or has not been taken. In the caregiver user interface 420, there is a second message 421, for the caregiver, informing the caregiver which medication needs to be taken by the patient 401. In FIG. 6, the status for the medication that needs to be taken is shown as Not Taken 424. Now, at 8:12 the patient 401 has indicated that they took this medication (e.g. by invoking the “Click when Taken” 414 directive) and the caregiver user interface 420 has been updated to indicate Taken 424A.

In some embodiments, an advertisement 416 is presented, possibly changing each time the caregiver user interface 420 is displayed or updated.

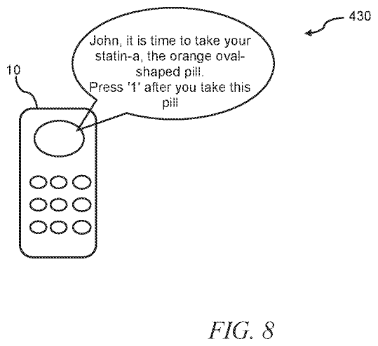

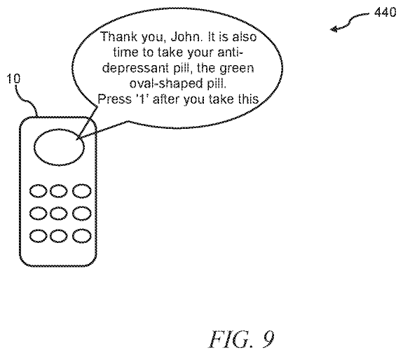

Referring to FIGS. 8 and 9, another exemplary method of informing a patient 401 that it is time to take their medication are shown. In this example, a voice call is made to the smartphone 10 (or any phone associated with the patient 401) and upon answering, a first audio message 430 is played, coming out of the smartphone's 10 earpiece or speaker saying that it is time to take the statin-A pill and instructing the patient 401 to “press ‘1’” after taking the statin-A pill. In FIG. 9, a second audio message 440 is played, coming out of the smartphone's 10 earpiece or speaker thanking the patient 401 and saying that it is time to take the antidepressant pill and instructing the patient 401 to “press ‘1’” after taking the antidepressant pill.

It is fully anticipated that a similar user interface be presented on a television (e.g. smart TV), perhaps using the audio portion of the television to emit the audio message and using the television remote control to confirm that the medication was taken (e.g. press the ‘1’ on your remote control). Likewise, in some embodiments, the request to take the medication is displayed in text on the television instead of audio.

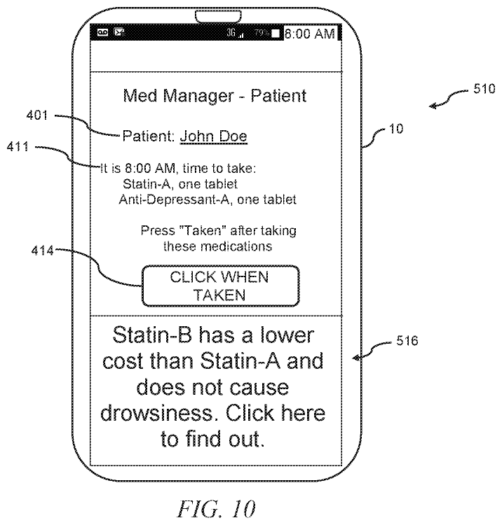

Referring to FIG. 10, an exemplary user interfaces for informing 510 a patient 401 that it is time to take a medication with an alternate therapy recommendation is shown. The exemplary user interfaces for informing 510 is similar to the exemplary patient user interface 410, except for the recommendation of alternate therapy 516. In the exemplary user interfaces for informing 510, the recommendation of alternate therapy 516 is related to one of the medications that the patient 401 is using, for example, the medication that the user needs to take at 8:00 AM. The patient 401 is using Statin-A and, in this fictitious example, it is known that Statin-A may cause drowsiness. To this, a pharmaceutical company that markets Statin-B directly advertises to the patient 401 that Statin-B does not cause drowsiness and informs the patient 401 that they can learn more by clicking on the recommendation of alternate therapy 516.

Based upon the laws of the United States and, potentially, other countries, it is often illegal to disclose any medical information without the patients consent. Therefore, it is not possible (or legal) for the system for presenting alternative therapy to disclose what medications or therapies the patient 401 is using to any pharmaceutical company. Instead of disclosing medications or therapies that the patient 401 is using, the system for presenting alternative therapy operates in an opposite fashion. The pharmaceutical company provides advertisement segments (text, images, video, music, commentator messages) and the advertisement segment(s) is/are associated with a medication or a class of medications. In the example of FIG. 10, the pharmaceutical company has subscribed to present the recommendation of alternate therapy 516 to patients 401 that have been prescribed Statin-A. Since the patient 401 is taking Statin-A, the recommendation of alternate therapy 516 from this pharmaceutical company is presented. In some embodiments, the pharmaceutical company subscribes to a classification of medications such as all statins, all pain relievers, any aspirin, etc. In some embodiments, the pharmaceutical company subscribes to a specific name of one or more medications such as statin-A or Statin-B, etc.

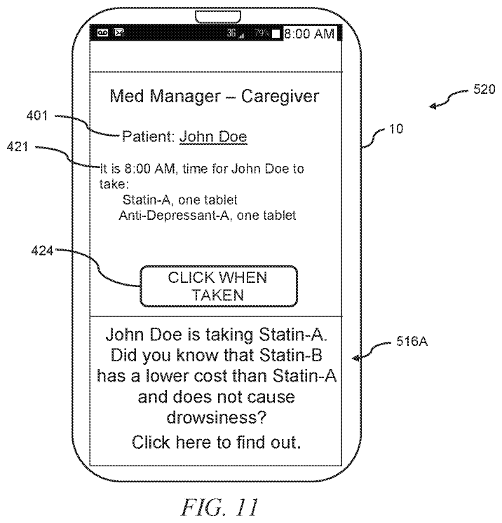

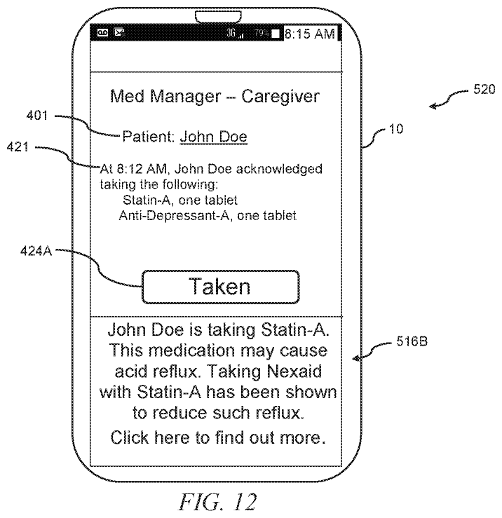

Referring to FIGS. 11 and 12, exemplary user interfaces for informing 520 a caregiver if and when the medication was taken medication having an alternate therapy recommendation are shown. As with FIGS. 6 and 7, many patients 401 have medical or aging issues that make it easy to miss taking their medications when required. The caregiver user interface 520 informs the caregiver (e.g. loved one, medical personnel) that the medication is due to be taken and has or has not been taken. In the caregiver user interface 520, there is a second message 521, for the caregiver, informing the caregiver which medication needs to be taken by the patient 401. In FIG. 11, the status for the medication that needs to be taken is shown as Not Taken 424. Now, at 8:12 the patient 401 has indicated that they took this medication (e.g. by invoking the “Click when Taken” directive 414 directive) and the caregiver user interface 520 has been updated to indicate Taken 424A as shown in FIG. 12. Note that in some embodiments, the user interface for informing a patient 410 is the same as the user interfaces for informing 520 a caregiver while in some embodiments, they are different.

In some embodiments, a recommendation of alternate therapy 516 is presented. As with FIG. 10, the pharmaceutical company provides advertisement segments (text, images, video, music, commentator messages) and the advertisement segment(s) is/are associated with a medication or a class of medications. In the example of FIGS. 11 and 12, the pharmaceutical company has subscribed to present the recommendation of alternate therapy 516 to patients 401 that have been prescribed Statin-A. Since the patient 401 is taking Statin-A or has Statin-A on their list of medications, the recommendation of an alternate therapy 516 from this pharmaceutical company is presented. In some embodiments, the pharmaceutical company subscribes to a classification of medications such as all statins, all pain relievers, any aspirin, etc. Although, in this example, the patient happens to be scheduled to take the Statin-A, there is no restriction as to which recommendation of an alternate therapy 516 is presented at any given time, but preferably, the recommendation of an alternate therapy 516 is related to a medication that the patient is currently taking. By related, in some embodiments, the recommendation of an alternate therapy 516 is keyed to a name of a medication that the patient is currently taking (e.g. Statin-A), in some embodiments the recommendation of an alternate therapy 516 is keyed to a classification of a medication that the patient is currently taking (e.g. any statin), while in some embodiments the recommendation of an alternate therapy 516 is keyed to a related medication to a medication that the patient is currently taking (e.g. taking a pain killer to reduce an effect of a different medication).

Since the recommendation of alternate therapy 516 is being presented to a caregiver (e.g. medical personnel, family member), in some embodiments, the content of the recommendation of alternate therapy 516A/516B is tailored to the recipient. For example, one recommendation of alternate therapy 516A is directed to the caregiver indicating that a lower cost alternative therapy is available while another recommendation of alternate therapy 516B suggests that if the patient 401 is experiencing a certain side effect when taking statin-A, taking of an advertised stomach medicine along with the statin-A may reduce this side effect. Having knowledge of who is using each user interface and the therapy (e.g. medicine) being used provides a powerful way to advertise alternate therapies that may have not been considered by the patient 401 and/or the caregivers in absence of such advertisements.

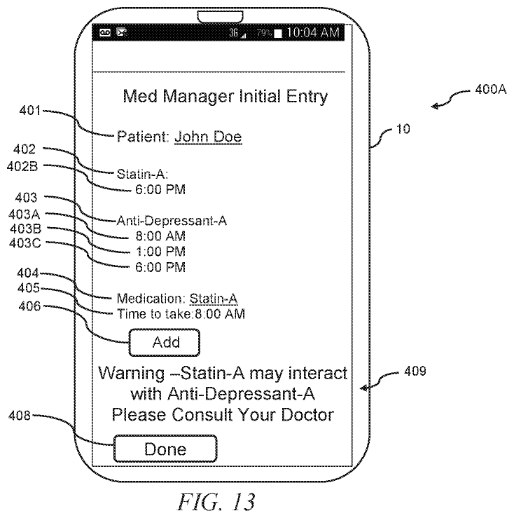

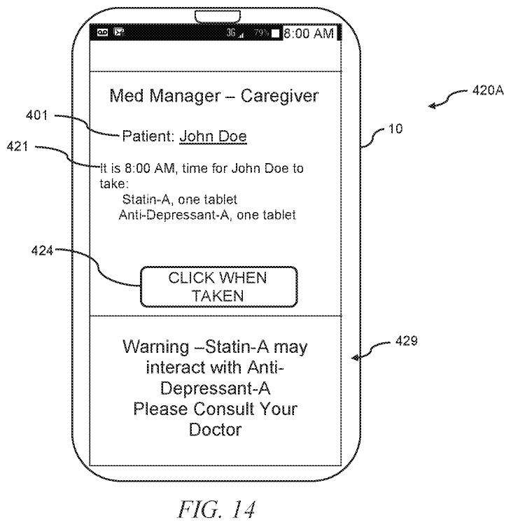

Referring to FIGS. 13 and 14, alternate exemplary user interfaces for entering a medication schedule 400A and for caregiver user interfaces 420 that include a drug interaction warnings 409/409A are shown. In some embodiments, drug interaction data is available to the system for presenting alternative therapy, for example, from a government provided database such as the national drug interaction directory 513 from a national organization such as the National Institute of Health (NIH) within the United States of America. Having drug interaction information, when the schedule for the patient 401 is established, used, or modified using the second exemplary user interface for entering a medication schedule 400A of FIG. 13, drug interaction checking is performed.

In the alternate exemplary user interface for entering a medication schedule 400A, some data has already been entered; Statin-A 402 and Anti-Depressant-A 403. The time of day to take each medication has also been entered; 6:00 PM 402B for the Statin-A 402 and 8:00 AM 403A, 1:00 PM 403B, and 6:00 PM 403C for the Anti-Depressant-A 403.

The user interface for entering medication data 400A has facilities to add/remove medications. For example, to add a new medication, a user enters the name of the medication 404, the time to take the medication 405 and then select the add feature 406. To remove a medication, the user right clicks on the medication and selects “delete.” To remove only one of the time entries (e.g. 8:00 AM 402A), the user right clicks on the time entry and selects delete. When finished entering medication information for the patient 401, the done feature 408 is invoked to save data and proceed with other user interface screens. In this user interface 400A, the user has entered Statin-A in the medication field 405 and time 8:00 AM in the time to take the medication field 406. Having drug interaction data, the system for presenting alternative therapy determines that there may be an interaction if Statin-A is taken at the same time as Anti-Depressant-A and presents a warning message 409 suggesting the caregiver or patient 401 contact their doctor to find out what interactions might occur.

In the alternate exemplary caregiver user interface 420A in FIG. 14, there is a message 421 for the caregiver, informing the caregiver which medication needs to be taken by the patient 401. In FIG. 14, the status for the medication that needs to be taken is shown as Not Taken 424. In some embodiments, an advertisement 416 is presented, possibly changing each time the caregiver user interface 420 is displayed or updated (see FIG. 6). In some embodiments, a recommendation of alternate therapy 516/516A/516B is presented, again, possibly changing each time the caregiver user interface 420 is displayed or updated (see FIG. 6). In embodiments that have access to drug interaction data, if the system for presenting alternative therapy determines that there may be an interaction, such as when Statin-A is taken at the same time as Anti-Depressant-A, the system presents a warning message 429 suggesting the caregiver or patient 401 contact their doctor to find out what interactions might occur.

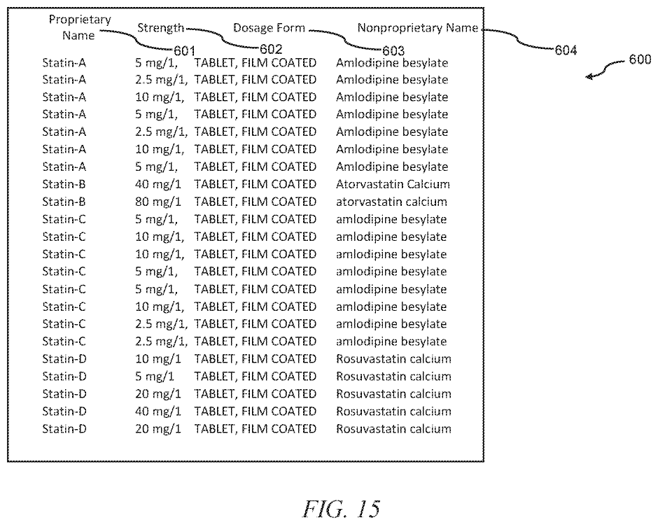

Referring to FIG. 15, exemplary data records 600 from the national drug code database as provided by the United States Food and Drug Administration (FDA) database are shown. These exemplary data records are extremely abbreviated as, for each medication/drug, in the national drug code database, there are many other fields such as generic name, various date fields, etc., all of which are not shown for clarity reasons. These exemplary data record 600 include the proprietary name 601 of each drug. As this is an exemplary data record, the proprietary names 601 have been replaced with the fictitious proprietary names as above to limit the use of registered trademarks in this application. Each exemplary data record also includes a strength 602 (e.g. 5 mg for one tablet), the dosage form 603, and a non-proprietary name 604.

The strength 602 is used by the system for presenting alternative therapies to match the medication that has been entered by, for example, the patient 401 or caregiver. The dosage form 603 is optionally used to describe the medication, for example, to describe the pill that needs to be taken as in FIG. 5.

The nonproprietary name 604 is also available to the system for presenting alternative therapies during presentation of alternate therapies in determining if/when to present recommendations of alternate therapy 516/516A/516B.

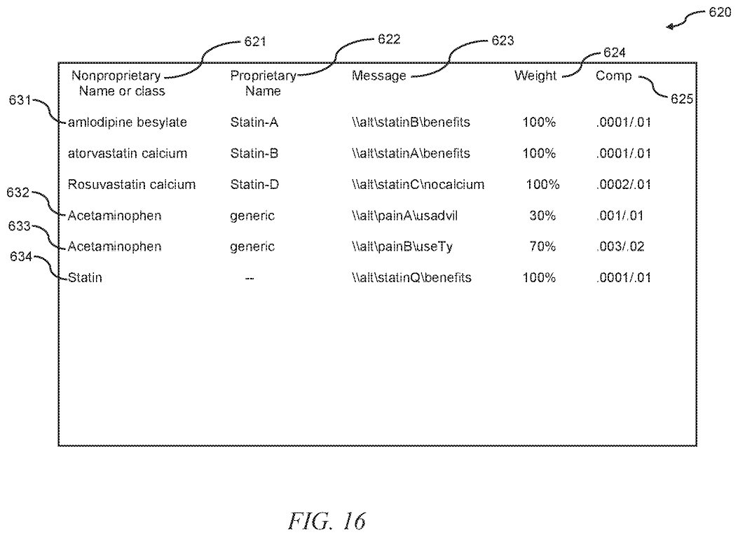

Referring to FIG. 16, an exemplary alternate therapy schedule 620 is shown. Each line of the alternate therapy schedule 620 includes a nonproprietary name 621 of the medication, a proprietary name 622, messages 623 which are, for example, pointers to files containing the recommendations of alternate therapy 516/516A/516B, and optionally a weight factor 624 and compensation factor 625. It is fully anticipated that in actual use, the alternate therapy schedule 620 have more or less data depending on pharmaceutical company requirements. For example, a pharmaceutical company may wish to only present recommendations of alternate therapy 516/516A/516B for a limited amount of time and, for such, the alternate therapy schedule 620 needs to include a start date and an end date (not shown for brevity and clarity).

The following are exemplary uses of the alternate therapy schedule 620. The first line 631 of the alternate therapy schedule 620 describes a medication, “amlodipine besylate Statin-A.” When the system for presenting alternative therapies finds amlodipine besylate or the proprietary name of Statin-A during generation of a user interface (see above), the system for presenting alternative therapies determines if there is an alternate therapy message that needs to be presented within that user interface. Finding a match of the nonproprietary name 621 and/or the proprietary name 622 in the first line 631, the system for presenting alternative therapies loads the message 623 (e.g. from a file \\alt\statinB\benefits stored in the alternate therapy data 504) and presents such recommendation of alternate therapy 516/516A/516B in the user interface. The fourth line 632 of the alternate therapy schedule 620 describes a medication, “Acetaminophen generic.” When the system for presenting alternative therapies finds Acetaminophen during generation of a user interface (see above), the system for presenting alternative therapies determines if there is an alternate therapy message that needs to be presented within that user interface. In the fourth line 622, there is a weight 624 of 30% which, in some embodiments, indicates that this recommendation is only presented 30% of possible times. Finding a match of the nonproprietary name 621 in the fourth line 632, the system for presenting alternative determines, based upon the weight 624 of 30% whether to load the message 623 (e.g. from a file \\alt\painA\usadvil stored in the alternate therapy data 504) and present such recommendation of alternate therapy 516/516A/516B in the user interface. Note that the fifth line 633 also lists Acetaminophen in the nonproprietary name 621. In this example, the system for presenting alternative therapies will present the message 623 (e.g. from a file \\alt\painA\usadvil stored in the alternate therapy data 504) 30% of the time and the message 623 (e.g. from a file \\alt\painB\useTy stored in the alternate therapy data 504) 70% of the time as there are two pharmaceutical companies that both have products that purportedly have advantages over generic Acetaminophen (e.g. less side effects, smaller tablets, coated tablets, lower cost).

The sixth line 634 is slightly different. In this, the non-proprietary name or class field 621 contains a classification of medications (statin) and there is no proprietary name 622. This means that this advertisement is eligible to be presented to a patient 401 or caregiver for the patient 401 that is taking any statin.

It is fully anticipated that the pharmaceutical companies will provide compensation to the providers of the system for presenting alternative therapies. In some embodiments, compensation is provided (billed) each time a recommendation of alternate therapy 516/516A/516B is presented as subscribed by the company. In some embodiments, compensation is provided (billed) each time a recommendation of alternate therapy 516/516A/516B is presented and the patient 401 or the caregiver clicks on the recommendation, as subscribed by the company. In some embodiments, both types of compensation are provided. In the alternate therapy schedule 620, the compensation field 625 of each line includes two numbers. A first number (e.g. 0.0001 of the first line 631) indicates compensation that is billed to the subscribing company each time a recommendation of alternate therapy 516/516A/516B is presented. A second number (e.g. 0.01 of the first line 631) indicates compensation that is billed to the subscribing company each time a recommendation of alternate therapy 516/516A/516B is selected (e.g. clicked on) by the patient 401 or caregiver.

It is fully anticipated that any recommendation of alternate therapy 516/516A/516B is presented in any order, sequential, random, when the medication is scheduled to be taken, etc.

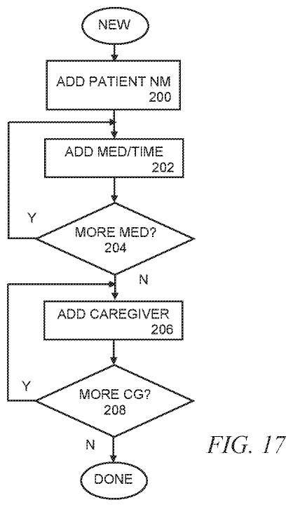

Referring to FIGS. 17-21, exemplary program flows of the system for presenting alternative therapies are shown. The program flows are shown for examples as it is well known to perform software tasks in many different ways achieving the same or similar outcomes.

It is anticipated that portions of the exemplary program flow execute on a user device such as a smartphone 10 while portions of the exemplary program flow execute on the server 500.

In this example, starting with FIG. 17, the flow starts when the system for presenting alternative therapy is provisioned with patients, medication schedules, and caregiver contacts. To start, the patient's name is obtained 200, for example by the patient 401 or caregiver typing the patient's name into a user interface. Next, a loop begins in which a medication and a time (or times) to take that medication are entered 202 until there are no more medications/times to enter 204. Again, it is anticipated that the medications and times are entered into a user interface. In some embodiments, using the camera 93 of the smartphone 10, the patient 401 or caregiver who is entering the information has the ability to capture a picture of the medicine label and the drug information is extracted from the label. Once all medications have been entered 204, a second loop begins where the caregiver contacts are entered 206 (e.g. caregiver names, phone numbers, email addresses). Once all caregivers have been entered 208, the provisioning is complete.

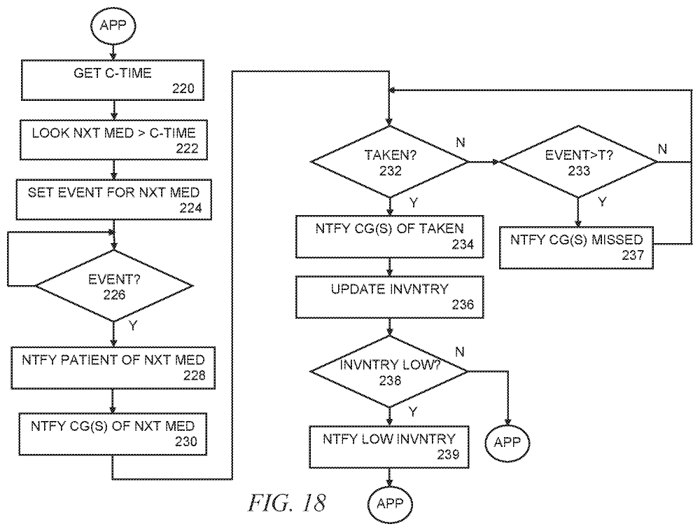

In FIG. 18, an exemplary program flow is shown for informing patients and caregivers when it is time to take medications. In this example, the patient uses an application to be notified and to acknowledge that the medication was taken. Program flow begins with obtaining the current time 220 then looking through the medication schedule to find which medication is to be taken next 222 (the first medication/time that is after the current time). An event is set to the time that the next medication needs to be taken 224.

When the event occurs 226, the patient is notified that it is time to take the next medication 228, for example, using the user interface for informing a patient 410 (see FIG. 5). At the same time, if the caregivers so desire (through settings), the caregiver(s) is/are notified that it is time for the patient 401 to take the next medication 230, for example, using the user interfaces for informing a caregiver 420 if and when the next medication was taken (see FIGS. 6 and 7). Note, the user interfaces for informing a caregiver 420 if and when the medication was taken will indicate that the next medication was not taken yet (Not Taken 424).

Now a loop 232 begins waiting for the patient 401 to indicate that they have taken the next medication. As the loop progresses, if there is no indication that the patient has taken their medication 232, a test 233 is made to determine if the time through this loop has exceeded a given amount of time, T. For example, if T is 30 minutes, has 30 minutes elapsed since the patient 401 was notified to take the next medication. If the test 233 determines that the time through this loop has exceeded a given amount of time, T, the caregiver(s) are notified that the patient 401 has missed their medication. Although the caregiver has the ability to adjust the time, T, it is preferred that this notification is not easily suppressed.

Note that it is anticipated that any or all notifications are made in any way such as through a text message, through an email message, through the caregiver's application, through a voice phone call, etc.

Once the patient 401 indicates that they have taken the next medication (e.g. selecting the “Click when Taken” directive 414—see FIG. 5), the caregiver(s) is/are notified that the patient 401 has taken the next medication 234, for example, using the user interfaces for informing a caregiver 420 (see FIGS. 6 and 7), text message, email, etc. Note, the user interfaces for informing a caregiver 420 will also indicate that the next medication was taken (Taken 424A).

In embodiments in which medication inventories are maintained, the inventory for the next medication is updated 236 (e.g. decremented by the number of pills taken). Now, if the inventory is low 238 (e.g. the number of remaining pills is less than a threshold that is set by the patient and/or caregiver(s)), notification is made 239 to the patient 401 and/or the caregiver(s) by, for example, a text message, phone call, message on one of the user interfaces, etc.

Now, the entire process of FIG. 18 repeats with the subsequent next medication.

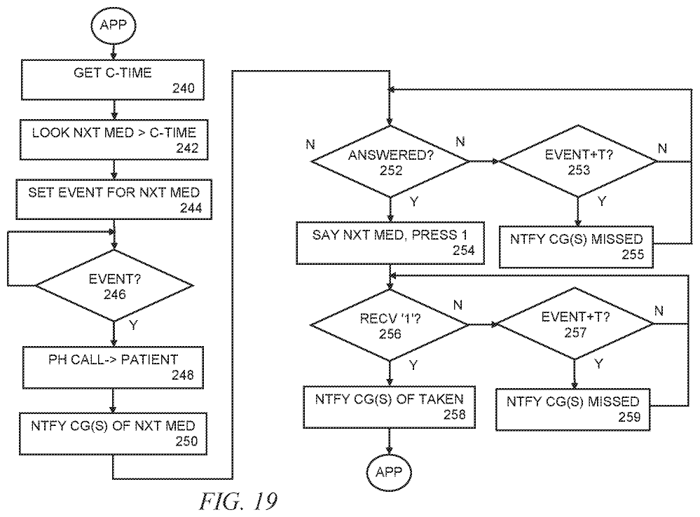

In FIG. 19, an exemplary program flow is shown for informing patients and caregivers when it is time to take medications. In this example, the patient is notified and acknowledges that the medication was taken through a voice phone call. Program flow begins with obtaining the current time 240 then looking through the medication schedule to find which medication is to be taken next 242 (the first medication/time that is after the current time). An event is set to the time that the next medication needs to be taken 244.

When the event occurs 246, the patient 401 is called and an audio message informs the patient 401 that it is time to take the next medication 248, for example, using the voice user interface for informing a patient 430/440 (see FIGS. 8 and 9). At the same time, the caregiver(s) is/are notified that it is time for the patient 401 to take the next medication 250, for example, using the user interfaces for informing a caregiver 420 if and when the next medication was taken (see FIGS. 6 and 7). Note, the user interfaces for informing a caregiver 420 will indicate that the next medication was not taken yet (Not Taken 424).

Now a loop 252 begins waiting for the patient 401 to answer their phone. As the loop progresses, if there is no indication that the patient has answered their phone 252, a test 253 is made to determine if the time through this loop has exceeded a given amount of time, T. For example, if T is 30 minutes, has 30 minutes elapsed since the patient 401 was notified to take the next medication. If the test 253 determines that the time through this loop has exceeded a given amount of time, T, the caregiver(s) are notified that the patient 401 has not answered their phone and has missed their medication. Although the caregiver has the ability to adjust the time, T, it is preferred that this notification is not easily suppressed.

Once answered, an audio message is emitted 254 (e.g. “it is time to take your Statin-A. Please press ‘1’ after taking your Statin-A”). Now the program waits for the patient 401 to press ‘1’ 256 to indicate that they have taken the next medication. As this loop progresses, if there is no indication that the patient has taken their medication 256, a test 257 is made to determine if the time through this loop has exceeded a given amount of time, T. For example, if T is 30 minutes, has 30 minutes elapsed since the patient 401 was notified to take the next medication. If the test 257 determines that the time through this loop has exceeded a given amount of time, T, the caregiver(s) are notified that the patient 401 has missed their medication. Although the caregiver has the ability to adjust the time, T, it is preferred that this notification is not easily suppressed.

Once the patient 401 indicates that they have taken the next medication (e.g. pressing ‘1’), the caregiver(s) is/are notified that the patient 401 has taken the next medication 258, for example, using the user interfaces for informing a caregiver 420 (see FIGS. 6 and 7). Note, the user interfaces for informing a caregiver 420 n will indicate that the next medication was taken (Taken 424A).

Now, the entire process of FIG. 19 repeats with the subsequent next medication.

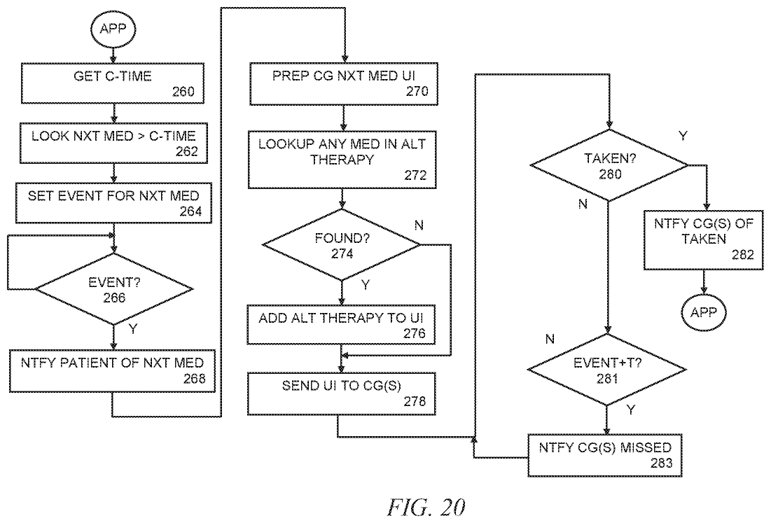

In FIG. 20, an exemplary program flow is shown for informing patients and caregivers when it is time to take medications. In this example, the patient 401 uses an application to be notified and the caregiver receives alternate therapy information when notified that it is time for the patient 401 to take the medication. Program flow begins with obtaining the current time 260 then looking through the medication schedule to find which medication is to be taken next 262 (the first medication/time that is after the current time). An event is set to the time that the next medication needs to be taken 264.

When the event occurs 266, the patient 401 is notified that it is time to take the next medication 268, for example, using the user interface for informing a patient 410 (see FIG. 5).

In this embodiment, if there are alternate therapies that are to be recommended, the proper recommendation is added to the user interface that is sent to the caregiver(s). This is done, in this example, by formatting 270 the caregiver user interface, for example, using the user interfaces for informing a caregiver 520 if and when the next medication was taken (see FIGS. 11 and 12). Now, either the next medication of any medication in the patient's list of medications is looked up 272 in the alternate therapy database 620. If the next medication is found 274 in the alternate therapy database 620, the informational message regarding the alternate therapy 516 is added 276 to the caregiver user interface 510 (see FIGS. 10 and 11). Note that it is anticipated, as previously described, that when there exists multiple alternate therapy 516 recommendations, there are algorithms to select the alternate therapy 516 is added 276 to the caregiver user interface 510 based upon various parameters such as random selection, revenues, round-robin, sequential, etc. Note also, if there are no informational messages regarding the alternate therapy 516, it is anticipated that, instead, a generic advertisement 416 is added to the caregiver user interface 510 (see FIGS. 10 and 11).

The caregiver user interface is then sent 278 to the caregiver(s) is/are to notify that it is time for the patient 401 to take the next medication along with the informational message regarding any alternate therapy 516 the is available, for example, using the user interfaces for informing a caregiver 520 if and when the next medication was taken (see FIGS. 11 and 12). Note, the user interfaces for informing a caregiver 520 if and when the medication was taken will indicate that the next medication was not taken yet (Not Taken 424).

Now a loop begins waiting for the patient 401 to indicate that they have taken 280 the next medication. As the loop progresses, if there is no indication that the patient has taken their medication 280, a test 281 is made to determine if the time through this loop has exceeded a given amount of time, T. For example, if T is 30 minutes, has 30 minutes elapsed since the patient 401 was notified to take the next medication. If the test 281 determines that the time through this loop has exceeded a given amount of time, T, the caregiver(s) are notified that the patient 401 has missed their medication. Although the caregiver has the ability to adjust the time, T, it is preferred that this notification is not easily suppressed.

Once the patient 401 indicates that they have taken 280 the next medication (e.g. selecting the “Click when Taken” directive 414—see FIG. 5), the caregiver(s) is/are notified that the patient 401 has taken the next medication 282, for example, using the user interfaces for informing a caregiver 520 (see FIGS. 11 and 12). Note, the user interfaces for informing a caregiver 520 will indicate that the next medication was taken (Taken 424A). Note also that the user interfaces for informing a caregiver 520 will still include the informational message regarding any alternate therapy 516.

Now, the entire process of FIG. 20 repeats with the subsequent next medication.

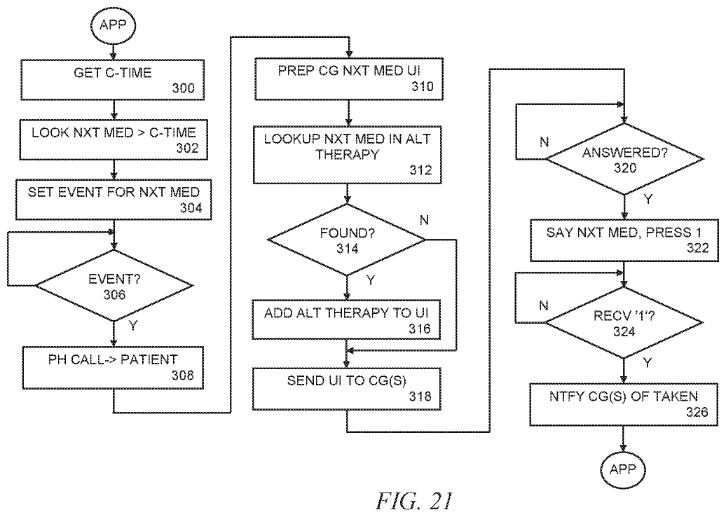

In FIG. 21, an exemplary program flow is shown for informing patients and caregivers when it is time to take medications. In this example, the patient is notified and acknowledges that the medication was taken through a voice phone call and the caregiver(s) are informed about possible alternative therapies. Program flow begins with obtaining the current time 300 then looking through the medication schedule to find which medication is to be taken next 302 (the first medication/time that is after the current time). An event is set to the time that the next medication needs to be taken 304.

When the event occurs 306, the patient 401 is called and an audio message informs the patient 401 that it is time to take the next medication 308, for example, using the voice user interface for informing a patient 430/440 (see FIGS. 8 and 9).

In this embodiment, if there are alternate therapies that are to be recommended, the proper recommendation is added to the user interface 520 that is sent to the caregiver(s). This is done, in this example, by formatting 310 the caregiver user interface, for example, using the user interfaces for informing a caregiver 520 if and when the next medication was taken (see FIGS. 11 and 12). Now, the next medication is looked up 312 in the alternate therapy database 620. If the next medication is found 314 in the alternate therapy database 620, the informational message regarding the alternate therapy 516 is added 316 to the caregiver user interface 520 (see FIGS. 11 and 12).

The caregiver user interface is then sent 318 to the caregiver(s) is/are to notify that it is time for the patient 401 to take the next medication along with the informational message regarding any alternate therapy 516 the is available, for example, using the user interfaces for informing a caregiver 520 if and when the next medication was taken (see FIGS. 11 and 12). Note, the user interfaces for informing a caregiver 520 will indicate that the next medication was not taken yet (Not Taken 424).

Now a loop 320 begins waiting for the patient 401 to answer their phone. Once answered, an audio message is emitted 322 (e.g. “it is time to take your Statin-A. Please press ‘1’ after taking your Statin-A”). Now the program waits for the patient 401 to press ‘1’ 324 to indicate that they have taken the next medication. Once the patient 401 indicates that they have taken the next medication (e.g. pressing ‘1’), the caregiver(s) is/are notified that the patient 401 has taken the next medication 326, for example, using the user interfaces for informing a caregiver 520 (see FIGS. 11 and 12). Note, the user interfaces for informing a caregiver 520 will indicate that the next medication was taken (Taken 424A).

Now, the entire process of FIG. 21 repeats with the subsequent next medication.

Again, any form of notification is anticipated.

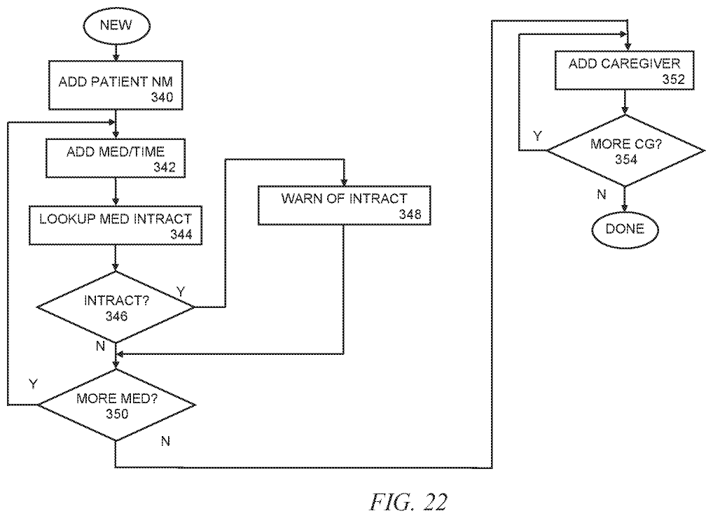

In FIG. 22, the flow starts when the system for presenting alternative therapy is provisioned with patients, medication schedules, and caregiver contacts, similar to FIG. 17 except this program flow includes drug interaction warnings. To start, the patient's name is obtained 340, for example by the patient 401 or caregiver typing the patient's name into a user interface. Next, a loop begins in which a medication and a time (or times) to take that medication are entered 342 until there are no more medications/times to enter 346. As each medication is entered 342, the medication name/composition is searched 344 in a medication interaction database to see if this medication has any adverse interactions with any other medication that was previously provisioned. Determination of drug interactions is performed using the national drug interaction directory 513 or equivalent. If this medication has the potential for any adverse interactions 346 with any other medication that was previously provisioned, the user entering the information is warned of the interaction 348 (e.g. asked to check with their medical practitioner).

Again, it is anticipated that the medications and times are entered into a user interface. In some embodiments, using the camera 93 of the smartphone 10, the patient 401 or caregiver who is entering the information has the ability to capture a picture of the medicine label and the drug information is extracted from the label.

Once all medications have been entered 350, a second loop begins where the caregiver contacts are entered 352 (e.g. caregiver names, phone numbers, email addresses). Once all caregivers have been entered 354, the provisioning is complete.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A system for presenting alternate therapies, the system comprising:
a server, the server having storage for storing medication information and schedules for a patient, the server connected to a data network;
software running on a caregiver's user device receives as inputs names of medications and schedules for taking the medications and the software running on the user device sends names of the medications and the schedules to the server, where the names of the medications and the schedules are stored in the storage;
software running on the server determines which of the medications is to be taken next and a time that the medication is to be taken;
at the time, the server notifies the patient that it is time to take the medication and the server transacts with the caregiver's user device and the caregiver's user device displays a caregiver user interface indicating that it is time for the patient to take the medication to inform the caregiver regarding the medication that is to be taken;
upon taking the medication, the server receives confirmation that the medication was taken and responsive to the confirmation, the server transacts with the caregiver's user device and the caregiver's user device displays a confirmation in the caregiver user interface to inform the caregiver that the medication was taken.

2. The system of claim 1, whereas the server notifies the patient that it is time to take the medication by sending a message to a patient's device and a patient user interface is displayed on the patient's device indicating the medication that is to be taken.

3. The system of claim 2, whereas if an alternate therapy for any medication in the medication information is available, information regarding the alternate therapy is sent from the server to the patient's device and/or to the caregiver, the information regarding the alternate therapy is displayed in the patient user interface.

4. The system of claim 2, whereas the server receives confirmation that the medication was taken by a confirmation input into the patient user interface that is sent to the server through the data network.

5. The system of claim 1, whereas the server notifies the patient that it is time to take the medication by making a voice call to a patient's phone and playing an audio message indicating the medication that is to be taken.

6. The system of claim 5, whereas the server receives confirmation that the medication was taken by a confirmation input into the patient's phone.

7. The system of claim 1, whereas if an alternate therapy for the medication is available, the software running on a caregiver's user device receives information regarding the alternate therapy and displays the information regarding the alternate therapy.

8. A method for providing information about an alternate therapy, the method comprising:
accepting inputs containing a name or classification of at least one medication;
searching a list of alternate therapies for each of the at least one medication;
for each of the at least one medication, if an alternate therapy is found in the list of alternate therapies, presenting information regarding the alternate therapy for that medication.

9. The method of claim 8, whereas the inputs containing a name of at least one medication further comprises a schedule of times for the patient to take each of the at least one medication.

10. The method of claim 9, further comprising the steps of monitoring the schedule of times for the patient to take each of the at least one medication and when it is time for the patient to take each of the at least one medication, notifying that it is time for the patient to take each of the at least one medication and if an alternate therapy was found in the list of alternate therapies for the at least one medication, presenting information regarding the alternate therapy for that medication.

11. The method of claim 9, further comprising the steps of monitoring the schedule of times for the patient to take each of the at least one medication and when it is time for the patient to take each of the at least one medication, notifying a caregiver that it is time for the patient to take each of the at least one medication and if an alternate therapy was found in the list of alternate therapies for the at least one medication, presenting information regarding the alternate therapy for that medication to the caregiver.

12. The method of claim 11, further comprising the steps of waiting for the patient to take each of the at least one medication and after the patient takes the at least one medication, notifying the caregiver that the patient has taken the at least one medication and if an alternate therapy was found in the list of alternate therapies for the at least one medication, presenting information regarding the alternate therapy for that medication to the caregiver.

13. A method for presenting alternate therapies, the method comprising:
receiving as inputs names of medications and schedules for taking the medications from a caregiver's user device;
sending the names of the medications and the schedules to a server through a data network, where the names of the medications and the schedules are stored in a storage;
determining which of the medications is to be taken next and determining a time that the medication is to be taken;
at the time, notifying the patient that it is time to take the medication and the server transacting with the caregiver's user device, operating the caregiver's user device to display a caregiver user interface indicating that it is time for the patient to take the medication;
if there is an alternate therapy for the medication in an alternate therapy list, the server transacting with the caregiver's user device, operating the caregiver's user device to display information regarding the alternate therapy in the caregiver user interface; and
upon taking the medication, receiving a confirmation from the patient that the medication was taken and the server receiving the confirmation and responsive to receiving the confirmation, the server transacting with the caregiver's user device, operating the caregiver's user device to display the confirmation in the caregiver user interface.

14. The method of claim 13, whereas the step of notifying the patient that it is time to take the medication is performed by sending a message to a patient's device causing a patient user interface to be displayed on the patient's device indicating the medication that is to be taken.

15. The method of claim 14, whereas if an alternate therapy for the medication is available, the step of notifying the patient that it is time to take the medication includes in the message information regarding the alternate therapy and displaying the information regarding the alternate therapy in the patient user interface.

16. The system of claim 15, whereas the step of receiving a confirmation from the patient that the medication was taken comprises invoking a confirmation directive at a patient's device and sending the confirmation from the patient's device to the server.

17. The method of claim 13, whereas the step of notifying the patient that it is time to take the medication is performed by making a voice call to a patient's phone and playing an audio message indicating the medication that is to be taken.

18. The method of claim 17, whereas the step of receiving a confirmation from the patient is initiated with the press of a key on the patient's phone.

19. The method of claim 13, further comprising after the step of confirmation from the patient, decrementing an inventory count of the medication that was taken.

20. The method of claim 19, further comprising after the step of decrementing an inventory count of the medication that was taken, if the inventory count of the medication that was taken is less than a predetermined count, the server transacting with the caregiver's user device, operating the caregiver's user device to display a warning regarding the inventory count of the medication that was taken in the caregiver user interface.
